(19) United States
(12) Patent Application Publication
(10) Pub. No.: US 2026/0069795 A1
(43) Pub. Date: Mar. 12, 2026

(54) NEEDLE UNIT FOR INJECTING DRUG AND SKIN TREATMENT DEVICE INCLUDING THE SAME

(71) Applicants: Suk Bae SEO, Seoul (KR); Ja Young KIM, Seoul (KR); Ho Joon SEO, Seoul (KR)

(72) Inventors: Suk Bae SEO, Seoul (KR); Ja Young KIM, Seoul (KR); Ho Joon SEO, Seoul (KR)

(21) Appl. No.: 19/333,814

(22) Filed: Sep. 19, 2025

Related U.S. Application Data
(63) Continuation of application No. PCT/KR2024/003109, filed on Mar. 11, 2024.

(30) Foreign Application Priority Data
Mar. 20, 2023 (KR) 10-2023-0035593

(51) Int. Cl. A61M 5/32; A61N 5/06; A61N 5/067

(52) U.S. Cl. CPC A61M 5/3298; A61M 5/3287; A61N 5/0616; A61N 5/067

(57) ABSTRACT

Provided is a needle unit for injecting a drug. The needle unit includes a cylinder, a plunger that reciprocates in an interior of the cylinder, a plurality of needles that are connected to the plunger and of which at least a partial area is disposed to be selectively exposed to an outside of the cylinder, and a pressing member that is interposed between the plunger and the needles and that spaces apart intervals between the needles exposed to an outside of the plunger. The drug is injected through an expansion hole formed in skin while intervals between tip ends of the needles are spaced apart from each other in a state in which the needles are inserted into the skin.

### CROSS-REFERENCE TO RELATED APPLICATIONS

The present application is a continuation of International Patent Application No. PCT/KR2024/003109, filed on Mar. 11, 2024, which is based upon and claims the benefit of priority to Korean Patent Application No. 10-2023-0035593 filed on Mar. 20, 2023. The disclosures of the above-listed applications are hereby incorporated by reference herein in their entirety.

### TECHNICAL FIELD

The present disclosure relates to a needle unit for injecting a drug, and a skin treatment device including the same.

### BACKGROUND

Recently, drug injection tips for scar treatment, hair growth treatment, bromhidrosis treatment, vascular treatment, fat reduction, wrinkle removal, skin elasticity recovery, sebum removal, and acne treatment have been developed.

A tip for injecting a drug may treat the skin by repeatedly inserting a plurality of needles into the skin to remove malignant tissue and form new tissue. Furthermore, the tip for injecting a drug removes collagen, elastic fibers, and the like of the skin by using heat generated by a current applied from a handpiece, promotes new formation, and improves pigmentation, acne scars, and wrinkles of the skin.

Meanwhile, the tip for injecting a drug may relieve pain that occurs in the skin and treat wounds that are generated in the skin in a process of inserting and discharging the plurality of needles into and from the skin, and may inject a drug into holes that are formed in the skin by the insertion and discharge of the plurality of needles. In this case, it is preferable to inject the drug into the widest possible area of the skin to enhance the efficacy of the drug.

However, according to a conventional tip for injecting a drug, because a diameter of a hole formed in the skin due to insertion and discharge of a plurality of needles is limited to a diameter of the needles, a drug injection range of the hole formed in the skin is narrow.

### DETAILED DESCRIPTION OF THE INVENTION

### Technical Problem

An aspect of the present disclosure provides a needle unit for injecting a drug, which may form an expansion hole into which a drug is injected after needles are inserted into skin so that a drug injection range of the skin may be expanded, and a skin treatment device including the same.

Problems to be solved by the present disclosure are not limited to the problems mentioned above, and other problems not mentioned will be clearly understood by those skilled in the art from the following description.

### Technical Solution

A skin treatment device including a needle unit for injecting a drug may include a cylinder, a plunger that reciprocates in an interior of the cylinder, a plurality of needles that are connected to the plunger and of which at least a partial area is disposed to be selectively exposed to an outside of the cylinder, and a pressing member that is interposed between the plunger and the needles and that spaces apart intervals between the needles exposed to an outside of the plunger, and the drug may be injected through an expansion hole formed in skin while intervals between tip ends of the needles are spaced apart from each other in a state in which the needles are inserted into the skin.

Furthermore, the pressing member may include a plate that is disposed below the plunger; interference parts that protrude below the plate and each having an accommodation recess, in which at least a portion of the corresponding needle is accommodated, and a coupling part that protrudes above the plate and configured to be selectively inserted into an extending portion protruding from a lower center of the plunger.

Furthermore, the cylinder may include at least one guide part that protrudes on an inner peripheral surface thereof to correspond to a movement direction and a movement range of the pressing member, and the plate may include a guide groove that is formed on an outer peripheral surface thereof such that at least a portion of the guide part is inserted thereinto, whereby reciprocation of the plate may be guided by the guide part in the interior of the cylinder.

Furthermore, the skin treatment device including the needle unit may further include an elastic member that is interposed between the plunger and the pressing member and that generates an elastic force such that the pressing member and the pressing member approach each other.

Furthermore, the skin treatment device including the needle unit may further include: a rod that connects an actuator elevating the pressing member, and the pressing member, when the rod lowers the pressing member, the elastic member connected to the pressing member may pull the plunger, and the plunger may be lowered until the plunger contacts an upper end of the guide part, and when the rod is further lowered after lowering of the plunger is completed, only the pressing member may be further lowered, whereby the interference parts space apart intervals between tip ends of the needles.

Furthermore, the needle may include: a first area; and a second area bent from the first area, and in a state in which a first area is received in the accommodation recess, the first area may be formed at the same angle as that of an inner peripheral surface of the accommodation recess, and the second area may be formed at a different angle from that of the inner peripheral surface of the accommodation recess.

Furthermore, the needles may be connected to be tilted from the plunger.

Furthermore, the cylinder may include a first space that is disposed above the plunger and a second space disposed below the plunger, and the plunger may include a channel that communicates the first space and the second space.

Furthermore, the pressing member may include a plurality of drug discharge holes that are formed to pass through the plate such that the drug supplied through the channel is discharged toward the skin from the second space.

Furthermore, the pressing member may include a plurality of needle insertion holes that are formed to pass through the plate such that the needles are connected to the plunger.

Furthermore, the skin treatment device including the needle unit may further include: a communication passage that is formed to communicate with the pressing member and the rod, and that supplies the drug to tip end areas of the needles, or through which a fiber laser for irradiating a laser beam to an epidermis layer of the skin is introduced and discharged.

### BRIEF DESCRIPTION OF DRAWINGS

FIG. 1 is a perspective view of a skin treatment device illustrating a state in which a needle unit for injecting a drug according to an embodiment of the present disclosure is installed in a handpiece.

FIG. 2 is a cross-sectional view illustrating a needle unit for injecting a drug according to an embodiment of the present disclosure.

FIG. 3 is a perspective view illustrating a state in which a pressing member of the needle unit for injecting a drug is coupled to one needle.

FIG. 4 is a perspective view illustrating a lower portion of a pressing member of the needle unit for injecting a drug.

FIG. 5 illustrates a cross-sectional view illustrating a state in which needles are partially exposed to an outside of a cylinder while a plunger of a needle unit for injecting a drug is lowered, and a plan view of skin.

FIG. 6 illustrates a cross-sectional view illustrating a state in which a drug is supplied to skin through a communication passage while a pressing member of a needle unit for injecting a drug is lowered so that intervals between needles are spaced apart, and a plan view of skin.

FIG. 7 illustrates a cross-sectional view illustrating a state in which a fiber laser is introduced through a communication passage and irradiates a laser beam to an epidermis layer of skin while a pressing member of a needle unit for injecting a drug is lowered so that intervals between needles are spaced apart, and a plan view of skin.

### MODE FOR CARRYING OUT THE INVENTION

The above and other aspects, features and advantages of the present disclosure will become apparent from embodiments to be described in detail in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

Unless otherwise defined, all terms (including technical and scientific terms) used herein have the same meaning as commonly understood by those skilled in the art to which the present disclosure pertains. The terms, such as those defined in commonly used dictionaries, should not be interpreted in an idealized or overly formal sense unless expressly so defined herein.

As illustrated in the drawings, spatially relative terms, such as “below”, “beneath”, “lower”, “above”, “upper”, and the like, may be used herein for ease of description to describe the relationship between one component and other components. It will be understood that the spatially relative terms are intended to encompass different orientations of the components in use or operation in addition to the orientation depicted in the drawing. For example, when inverting a component shown in the drawings, a component described as “below” or “beneath” of another component may be placed “above” another element. Thus, the exemplary term “below” may include both downward and upward directions. The components may also be oriented in different directions, and thus the spatially relative terms may be interpreted depending on orientation.

Before the description of the present disclosure, in various embodiments, components having the same configuration will be denoted by the same reference numerals and will be representatively described in one embodiment, and in other embodiments, only configurations that are different from those of the one embodiment will be described.

In indicating directions of the present disclosure, an upper side of a drawing will be defined as an upper side, a lower side of the drawing will be defined as a lower side, and an upward/downward direction of the drawing will be defined as an upward/downward direction.

Hereinafter, embodiments of the present disclosure will be described in detail with reference to accompanying drawings.

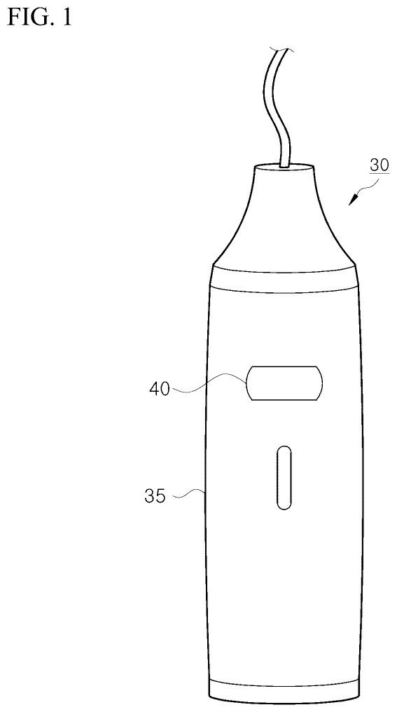

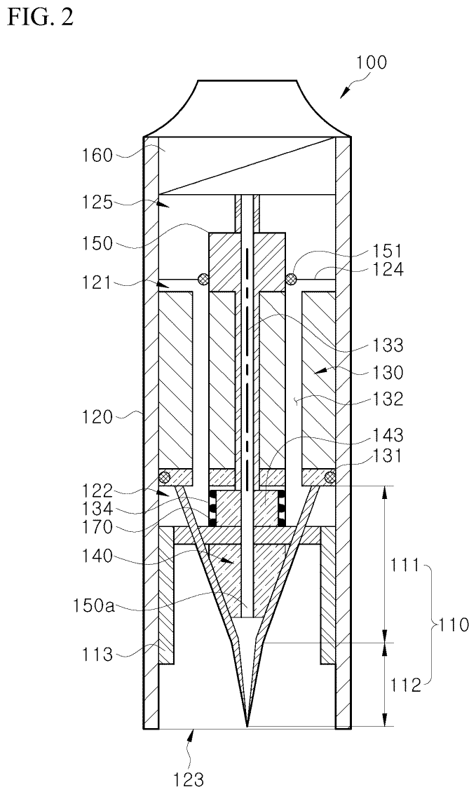

FIG. 1 is a perspective view illustrating a skin treatment device provided with needles for injecting a drug according to an embodiment of the present disclosure, and FIG. 2 is a cross-sectional view illustrating a skin treatment device provided with needles for injecting a drug.

A needle unit 100 for injecting a drug according to an embodiment of the present disclosure may be separably coupled to a lower end of a skin treatment device 30.

A handpiece 35 may be used as a handle gripped by an operator. Specifically, when the skin treatment device 30 is gripped by an operator and contacts a skin of a subject and the needles 110 are partially inserted into the skin, an expansion hole 11 may be formed in the skin 10 by spacing apart intervals of tip ends of the needles 110, so that a drug applied onto the skin 10 or a drug supplied onto the skin 10 from an interior of the skin treatment device 30 may be easily introduced into the expansion hole 11 of the subject. Of course, the operator may change a target portion of the skin 10 of the subject, into which the drug is injected, while moving the handpiece 35.

Furthermore, the skin treatment device 30 may be provided with a drug supply part (not illustrated) that supplies a drug 20 to tip end areas of the needles 110, and a power supply (not illustrated) that selectively applies current to the needles 110. The drug supply part and the power supply may be controlled by a controller 40.

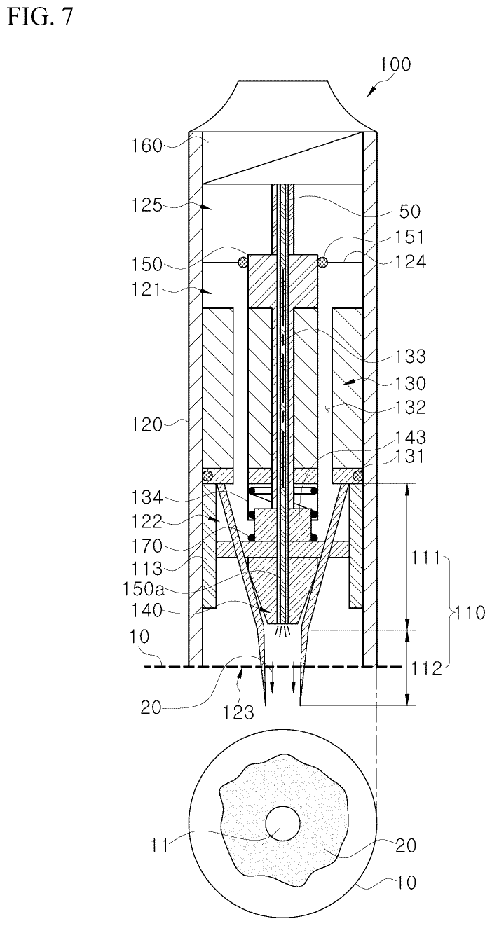

As an example, the controller 40 is provided in the handpiece, and when the needles 110 are inserted into the skin 10, the drug supply part may be controlled to supply the drug to the tip end areas of the needles 110 through a communication passage 150a (see FIG. 2) formed to communicate along a central axis of a pressing member 140 and a rod 150, or the power supply may be controlled to apply current to the needles 110. Furthermore, the controller 40 may perform a control such that a fiber laser 50 (see FIG. 7) that is introduced and discharged through the communication passage 150a irradiates a laser beam to an epidermis layer of the skin 10. Here, the drug or the fiber laser may be selectively introduced into the communication passage 150a. Furthermore, the communication passage 150a may be selectively provided. Furthermore, a plurality of communication passages may be provided.

Furthermore, when the needles 110 are discharged from the skin 10, the controller 40 may control the drug supply part to stop supplying the drug to the needles 110 or the power supply to stop applying current to the needles 110.

The needle unit 100 for injecting a drug according to an embodiment of the present disclosure includes a cylinder 120, a plunger 130, the needles 110, and the pressing member 140.

First, the cylinder 120 is separably coupled to a lower end of the handpiece 35. Here, although not illustrated as another embodiment, the needle unit 100 for injecting a drug may not be separably coupled to the handpiece 35, and an exterior shape of the cylinder 120 may be formed in a shape of the handpiece to have a function of the handpiece.

The cylinder 120 defines an accommodation space such that the plunger 130 may reciprocate, and the accommodation space includes a first space 121 that is disposed above the plunger 130 and a second space 122 that is disposed therebelow. Of course, the first space 121 and the second space 122 may have a cross-sectional area or a volume that is relatively changed due to reciprocation of the plunger 130.

The cylinder 120 has a shape in which an upper side of the first space 121 is closed, and a tip end of a lower side (or an outer side) of the second space 122 has an open structure. That is, an opening 123 is formed at a lower side of the second space 122. Of course, although not illustrated in the drawings, the opening 123 may allow only entry and exit of the needles 110, and a remaining area may be applied as a partially closed structure. In the present embodiment, the opening 123 is formed at a lower end of the cylinder 120, and a periphery of the opening 123 may contact the skin 10.

A shielding part 124 that protrudes inward in a flange shape from an inner peripheral surface of the cylinder 120 may be provided on the first space 121 of the cylinder 120. A through-hole may be formed in the shielding part 124 such that the rod 150 may be moved at a center of an interior thereof, and an O-ring 151 for maintaining airtightness may be provided between an inner peripheral surface of the shielding part 124 and the rod 150. Accordingly, the cylinder 120 is provided with a separated third space 125 at an upper portion of the first space 121 with respect to the shielding part 124.

The third space 125 is provided with an actuator 160, and the rod 150 provides a function of transmitting kinetic energy generated from the actuator 160 to the plunger 130. In this case, an upper area of the rod 150 may be disposed to span between the first space 121 and the third space 125, and a lower area thereof may be disposed to pass through a center of an interior of the plunger 130.

Furthermore, the plunger 130 is disposed to reciprocate in an upward/downward direction in response to movement of the rod 150. Although not illustrated in the drawings, a guide structure (not illustrated) that limits a movement direction or distance between the cylinder 120 and the rod 150 may be provided. The plunger 130 may be formed in a size corresponding to a diameter of the cylinder 120, and an O-ring 131 for maintaining airtightness may also be provided between the plunger 130 and the cylinder 120.

A channel 132 that passes through the plunger 130 in upward and downward directions may be formed in the interior of the plunger 130. The channel 132 may provide a function of serving as a passage that communicates the first space 121 and the second space 122, and it is preferable that a plurality of channels 132 be formed in a preset pattern.

The plurality of needles 110 and the pressing member 140 are disposed in the second space 122.

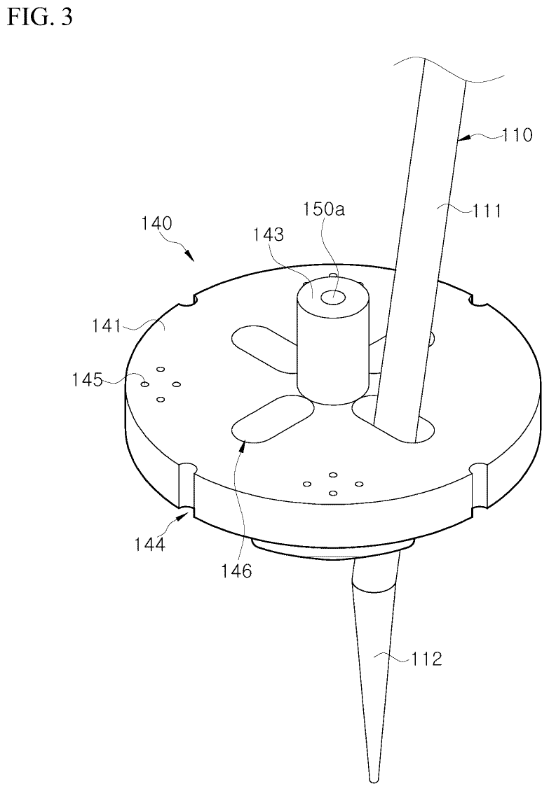

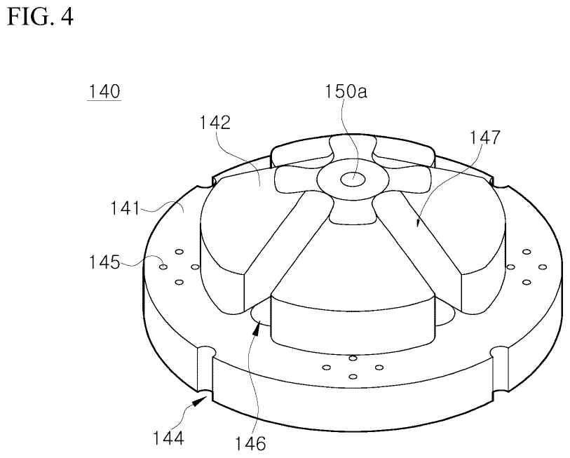

FIG. 3 is a perspective view illustrating a state in which the pressing member of the needle unit for injecting a drug is coupled to one needle, and FIG. 4 is a perspective view illustrating a lower portion of the pressing member of the needle unit for injecting a drug.

Referring to FIGS. 2 to 4, the needles 110 are coupled to be adjacent to a peripheral area at a lower end of the plunger 130. In this case, the needles 110 may be coupled to be tilted from the plunger 130. This is to allow the needles 110, the intervals of which will be widened later, to maintain a coupled state with the plunger 130.

The needles 110 may be coupled through a holder 135 that is provided at a lower end of the plunger 130.

Next, a plurality of, or at least two needles 110 are provided. In the present embodiment, a structure in which four needles 110 are disposed at equal intervals or equal angles with each other will be described as an example.

The four needles 110 may be disposed in directions that are perpendicular to each other between adjacent needles 110 at a lower peripheral area of the plunger 130. Of course, positions of the needles 110 are not limited to the directions or angles, as long as tip ends of the needles 110 may be spaced apart from each other.

The needle 110 includes a first area 111 that is disposed on an upper side, and a second area 112 that is disposed below the first area 111. In this case, an inclination angle of the first area 111 and an inclination angle of the second area 112 are formed differently, and the needle 110 is formed at an inclination angle that, with respect to an imaginary central axis 133 of the plunger 130, becomes close to the central axis 133 as it goes from the first area 111 to the second area 112. That is, the first area 111 of the needle 110 is disposed to have a relatively large inclination angle from the central axis 133, and the second area 112 is disposed to have a relatively small inclination angle from the central axis 133. Furthermore, a tip end of the second area 112 is the sharpest area, and the tip ends of the needles 110 are disposed to be closely attached to each other so as to face one point. A description that tip ends of the needles 110 are spaced apart from each other will be given later.

And, the pressing member 140 is interposed between the plunger 130 and the needles 110, and is disposed to maintain the same height with respect to each of the needles 110.

The pressing member 140 includes a plate 141, an interference part 142, and a coupling part 143.

The plate 141 may have a substantially circular disk shape, and a plurality of guide grooves 144 may be formed on an outer peripheral surface thereof. The guide groove 144 is disposed to correspond to a guide part 113 that protrudes from an inner peripheral surface of the second space 122 of the cylinder 120 along a movement direction of the plunger 130, and the pressing member 140 is guided to reciprocate along the guide part 113. Accordingly, a movement direction and a movement range of the pressing member 140 may be set by the guide part 113.

Here, an upper end of the guide part 113 may be disposed to interfere with a lower end of the plunger 130. Accordingly, when the plunger 130 contacts the guide part 113 while being lowered, movement thereof may be stopped.

Furthermore, a plurality of drug discharge holes 145 are formed on the plate 141, and four needle insertion holes 146 are formed such that the needles 110 may pass therethrough, respectively.

The coupling part 143 protrudes from an upper portion of the plate 141. The coupling part 143 may be selectively inserted into an interior of an extending portion 134 that protrudes downward from a lower end of a central area of the plunger 130. Accordingly, the coupling part 143 may have a shape corresponding to the extending portion 134.

An elastic member 170 is provided between the coupling part 143 and the extending portion 134. The elastic member 170 generates an elastic force such that the plunger 130 and the pressing member 140 approach each other. Accordingly, in a state in which a force in response to movement of the plunger 130 or the pressing member 140 is not applied, the coupling part 143 is disposed to be inserted into or closely attached to the interior of the extending portion 134 by the elastic force of the elastic member 170.

Accordingly, the pressing member 140 may be directly connected to the rod 150 to be moved in response to movement of the rod 150, and may be indirectly connected to the plunger 130 through the elastic member 170.

An interference part 142 protrudes from a lower portion of the plate 141. An accommodation recess 147 in which the first area 111 of each of the needles 110 is at least partially accommodated is formed in the interference part 142. The interference part 142 may be formed in a triangular or trapezoidal cross-sectional shape in a shape, of which a cross-sectional area becomes smaller as it goes downward with respect to the imaginary central axis 133 of the plunger 130.

In this case, the needle insertion hole 146 and the accommodation recess 147 may be disposed to at least partially overlap each other on a vertical cross-section. Accordingly, the intervals of the needles 110 that passed through the needle insertion hole 146 may be changed in response to elevation of the interference parts while the first areas 111 are partially inserted into the accommodation recesses 147.

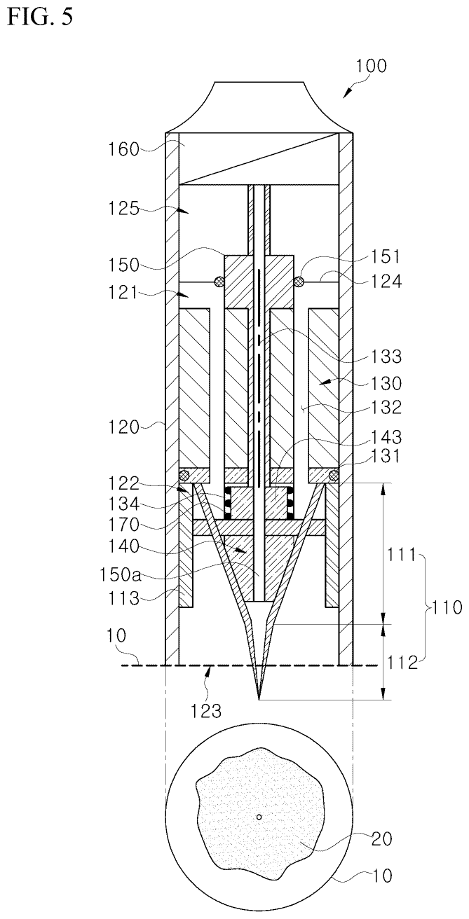

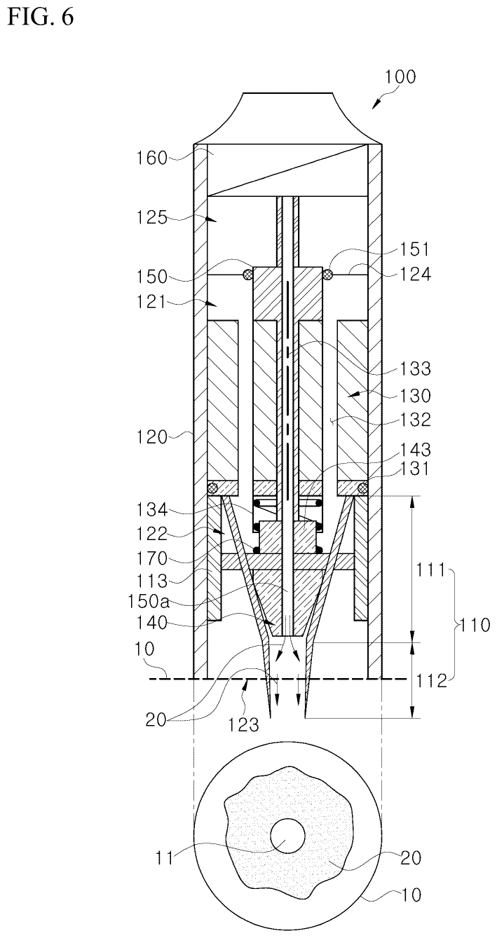

FIG. 5 is a cross-sectional view illustrating a state in which the needles are partially exposed to the outside of the cylinder while the plunger of the needle unit for injecting a drug is lowered and a plan view of the skin, and FIG. 6 is a cross-sectional view illustrating a state in which intervals between the needles are spaced apart while the pressing member of the needle unit for injecting a drug is lowered and a plan view of the skin.

Referring to FIGS. 5 and 6, an operation of the needle unit 100 for injecting a drug starts from movement of the rod 150.

When the rod 150 is lowered in a state in which the cylinder 120 is closely attached to the skin 10, the pressing member 140 pulls the plunger 130 downward to an upper end of the guide part 113 while being lowered. Then, when the plunger 130 contacts an upper end of the guide part 113, the tip ends of the needles 110 protrude to the outside of the opening 123, and are inserted into the skin 10.

When the plunger 130 contacts the upper end of the guide part 113, lowering of the plunger 130 is stopped, and only the pressing member 140 is in a state of being able to be further lowered.

The plunger 130 is connected to the pressing member 140 by the elastic member 170.

FIG. 5 illustrates a state in which the plunger 130 has been moved together with the pressing member 140 to contact the upper end of the guide part 113.

And, FIG. 6 illustrates a state in which lowering of the plunger 130 is stopped, and only the pressing member 140 is further lowered. The pressing member 140 is moved downward at the same time when the rod 150 is lowered, and in this case, because lowering of the plunger 130 is stopped, the needles 110 are not further lowered in the skin 10.

Furthermore, when the pressing member 140 is further lowered as the rod 150 is further lowered, the interference parts 142 may be rotated or elastically deformed in a direction in which the tip ends of the needles 110 become more distant from each other while pressing the first areas 111 of the needles 110. Because the needles 110 are coupled by the holder 135 at a lower end of the plunger 130, they may be tilted with respect to the plunger 130 or elastically deformed (for example, curved).

Then, tip ends of the second areas 112 of the needles 110 may be disposed to be spaced apart from each other. Spacing of the tip ends of the needles 110 corresponds to forming an expansion hole 11 in a state in which the needles 110 are inserted into the skin 10. That is, after the needles 110 are inserted into the skin 10, the needles 110 that have been gathered at one point are spaced apart from each other to form the expansion hole 11.

In a more detailed description of an operation of the needle unit 100 for injecting a drug according to an embodiment of the present disclosure, as illustrated in FIG. 5, the plunger 130 is moved downward in a state in which the cylinder 120 is closely attached to the skin 10. In this case, the needles 110 protrude from the second space 122 of the cylinder 120 to the skin 10, and the second areas 112 of the needles 110 are inserted into the skin 10 to form an invasive hole in the skin 10.

Next, as illustrated in FIG. 6, the plunger 130 is further moved downward. In this case, in a state in which the second areas 112 of the needles 110 are inserted into the skin 10, an expanded expansion hole 11 is formed in the skin 10 while the pressing member 140 spaces intervals between the needles 110.

In this way, because a negative pressure is formed in the second space 122 when gas of the second space 122 flows to the first space 121 through the channel 132 while the plunger 130 is moved downward, the skin 10 may be suctioned to the second space 122 of the cylinder 120. As a result, because a surface of the skin 10 suctioned into the second space 122 of the cylinder 120 becomes flat, the needles 110 may be easily inserted.

Furthermore, when the pressing member 140 is further lowered, as illustrated in FIG. 6, the expansion hole 11 is formed in the skin 10 while tip ends of the second areas 112 of the needles 110 are spaced apart from each other in a state in which the second areas 112 of the needles 110 are inserted into the invasive hole.

In a state in which the needles 110 are inserted into the skin 10 to form the expansion hole 11, the drug 20 applied onto the skin 10 is injected deep into the skin 10 through the expansion hole 11. In this case, the controller 40 may control the drug supply part to supply the drug 20 to the skin 10 located at tip end areas of the needles 110 through the communication passage 150a. Here, the controller 40 may control the drug supply part to supply the drug 20 to the first space 121 instead of the communication passage 150a, to supply the drug 20 to the skin 10 through the drug discharge hole 145 via the channel 132 and the second space 122.

Furthermore, in a state in which the needles 110 are inserted into the skin 10 to form the expansion hole 11, the controller 40 may control a power supply to apply current to the needles 110. By using heat generated by the current applied to the needles 110, collagen and elastic fibers of the skin may be removed, a new formation of the skin may be promoted, and pigmentation of the skin, acne scars, and wrinkles may be improved.

Here, as illustrated in FIG. 6, the drug 20 may not be additionally supplied to the skin 10 through the communication passage 150a, but as illustrated in FIG. 7, the fiber laser 50 may be introduced through the communication passage 150a in a state in which the needles 110 are spaced apart from each other while the pressing member 140 of the needle unit 100 for injecting a drug is lowered, so that an epidermis layer of the skin 10 may be protected by irradiating a laser beam to the epidermis layer.

Meanwhile, in a state in which the cylinder 120 is closely attached to the skin 10, the plunger 130 is moved upward. In this case, while intervals between the needles 110 are narrowed again, the needles 110 are closely attached to each other and discharged from the skin 10, and the expansion hole 11 formed in the skin 10 is exposed on a surface of the skin 10.

Due to the expansion hole 11 exposed in this way, while the plunger 130 is moved upward, a positive pressure is formed in the second space 122 while gas of the first space 121 of the cylinder 120 flows to the second space 122 through the channel 132, and thus, the drug 20 applied to the skin 10 or the drug 20 accommodated in the needles 110 may be injected deep into the expansion hole 11.

Accordingly, according to the needle unit for injecting a drug of an embodiment of the present disclosure, as the pressing member expands the intervals between the needles after the needles are inserted into the skin, an expanded expansion hole may be formed in the skin and the drug may be injected, so that a drug injection range of the skin may be expanded.

Meanwhile, in the above-described embodiment, it is illustrated that tip ends of the plurality of needles are disposed to be closely attached to each other to form one point on an imaginary central axis of the plunger, and the tip ends of the needles are closely attached to each other or spaced apart from each other as the pressing member is moved, but the present disclosure is not limited thereto, and the plurality of needles may be disposed at a lower end of the plunger to be spaced apart from each other, so that the needles may form expansion holes in the skin in response to the movement and operation of the pressing member, respectively.

Although an embodiment of the present disclosure are described with reference to the accompanying drawings, it will be understood by those skilled in the art to which the present disclosure pertains that the present disclosure can be carried out in other detailed forms without changing the scope and spirit or the essential features of the present disclosure. Therefore, the above-described embodiments are exemplary in all aspects, and should be construed not to be restrictive.

1. A needle unit for injecting a drug, the needle unit comprising:
a cylinder;
a plunger configured to reciprocate in an interior of the cylinder;
a plurality of needles connected to the plunger and of which at least a partial area is disposed to be selectively exposed to an outside of the cylinder; and
a pressing member interposed between the plunger and the needles and configured to space apart intervals between the needles exposed to an outside of the plunger,
wherein the drug is injected through an expansion hole formed in skin while intervals between tip ends of the needles are spaced apart from each other in a state in which the needles are inserted into the skin.

2. The needle unit of claim 1, wherein the pressing member includes:
a plate disposed below the plunger;
interference parts protruding below the plate and each having an accommodation recess, in which at least a portion of the corresponding needle is accommodated; and
a coupling part protruding above the plate and configured to be selectively inserted into an extending portion protruding from a lower center of the plunger.

3. The needle unit of claim 2, wherein the cylinder includes at least one guide part protruding on an inner peripheral surface thereof to correspond to a movement direction and a movement range of the pressing member,
wherein the plate includes a guide groove formed on an outer peripheral surface thereof such that at least a portion of the guide part is inserted thereinto, whereby reciprocation of the plate is guided by the guide part in the interior of the cylinder.

4. The needle unit of claim 3, further comprising:
an elastic member interposed between the plunger and the pressing member and configured to generate an elastic force such that the pressing member and the pressing member approach each other.

5. The needle unit of claim 4, further comprising:
a rod connecting an actuator elevating the pressing member, and the pressing member,
wherein when the rod lowers the pressing member, the elastic member connected to the pressing member pulls the plunger, and the plunger is lowered until the plunger contacts an upper end of the guide part, and
wherein when the rod is further lowered after lowering of the plunger is completed, only the pressing member is further lowered, whereby the interference parts space apart intervals between the tip ends of the needles.

6. The needle unit of claim 5, wherein the needle includes:
a first area; and
a second area bent from the first area, and
wherein in a state in which a first area is received in the accommodation recess, the first area is formed at the same angle as that of an inner peripheral surface of the accommodation recess, and the second area is formed at a different angle from that of the inner peripheral surface of the accommodation recess.

7. The needle unit of claim 5, wherein the needles are connected to be tilted from the plunger.

8. The needle unit of claim 2, wherein the cylinder includes a first space disposed above the plunger and a second space disposed below the plunger, and
wherein the plunger includes a channel communicating the first space and the second space.

9. The needle unit of claim 8, wherein the pressing member includes:
a plurality of drug discharge holes formed to pass through the plate such that the drug supplied through the channel is discharged toward the skin from the second space.

10. The needle unit of claim 2, wherein the pressing member includes:
a plurality of needle insertion holes formed to pass through the plate such that the needles are connected to the plunger.

11. The needle unit of claim 5, further comprising:
a communication passage formed to communicate with the pressing member and the rod, and configured to supply the drug to tip end areas of the needles, or through which a fiber laser for irradiating a laser beam to an epidermis layer of the skin is introduced and discharged.

12. A skin treatment device including the needle unit of claim 1.
